(19) United States
(12) Patent Application Publication
(10) Pub. No.: US 2010/0305130 A1
(43) Pub. Date: Dec. 2, 2010

(54) NASAL SPRAY DEVICE AND METHOD OF USE THEREOF

(76) Inventor: Thomas PHILLIPS, Watkinsville, GA (US)

Correspondence Address: ANDREWS KURTH LLP, 1350 I STREET, N.W., SUITE 1100, WASHINGTON, DC, 20005, US

(21) Appl. No.: 12/475,662

(22) Filed: Jun. 1, 2009

(51) Int. Cl. A61K 31/5375; A61K 31/24; A61K 31/16; A61K 31/46; A61P 25/20; A61M 11/00

(52) U.S. Cl. 514/239.2; 514/535; 514/626; 514/304; 128/200.14

(57) ABSTRACT

A single-use sprayer for nasal anesthesia is disclosed. The single-use sprayer includes a single dose of anesthetic and a delivery system adapted to dispense the single dose of anesthetic into a nostril. The single-use sprayer prevents patient-to-patient contamination, is easy to use and allows for self-administration, and has a simple, disposable configuration so as to lower the production cost.

### FIELD

The present invention relates to a nasal spray device, and more particularly; to a disposable, single-use sprayer for nasal anesthesia

### BACKGROUND

Nasal anesthesia is a common procedure performed in many medical office. For example, nasal anesthesia is used in nasopharyngoscopy, oropharyngoscopy, laryngoscopy, nasal antroscopy, examination of nasal turbinates, trans-nasal maxillary antroscopy, trans-nasal sphenoidoscopy, trans-nasal ethmoidoscopy and trans-nasal eustacionoscopy. Presently, doctors use an atomizing sprayer, which contains multiple doses and is re-usable. Because the device is re-used, it must be cleaned and/or sterilized between uses. If the device is not cleaned properly, infections could be transferred between patients. Further, the content of a multiple use nasal anesthetic sprayer is not used up at the expiration date, the un-used doses have to be discarded and hence increases cost of medical care.

### SUMMARY

One aspect of the present invention relates to a single-use sprayer for nasal anesthesia, comprising a single dose of anesthetic and a delivery system adapted to dispense the single dose of anesthetic into a nostril.

In one embodiment, the anesthetic is a topical anesthetic selected from the group consisting of benzocaine, butamben, dibucaine, lidocaine, oxybuprocaine, pramoxine, proparacaine, proxymetacaine, cocaine, and tetracaine,

In a related embodiment, the topical anesthetic comprises lidocaine, preferably 2% lidocaine or 4% lidocaine.

In another related embodiment, the topical anesthetic comprises a mixture of oxymetazoline and lidocaine, preferably 0.05% oxymetazoline and 2% or 4% lidocaine.

In another related embodiment, the topical anesthetic comprises a mixture of tetracaine and oxymetazoline, preferably I % tetracaine in oxymetazoline.

In another related embodiment, the topical anesthetic comprises cocaine, preferably 4% cocaine.

In another embodiment, in the delivery system comprises a squeeze bottle containing the single dose of anesthetic in solution form, wherein the squeeze bottle is sealed with a tamper proof cap.

In another embodiment, the delivery system comprises a pressurized container that contains the single-dose anesthetic, a one-way valve, and an applicator, wherein the one-way valve can be manually open to dispense the anesthetic in a nostril through the applicator,

In a related embodiment, the applicator has a substantially frustoconical profile with curved end edges to facilitate insertion into the nostril.

In another embodiment, the delivery system comprises a container that contains the single-dose anesthetic; and a manually operable pump for dispensing the anesthetic in a nostril through an applicator.

Another aspect of the present invention relates to a kit for nasal anesthesia. The kit contains at least one single-use sprayer comprising a single dose of anesthetic and a delivery system; and an instruction describing how to use the sprayer.

Another aspect of the present invention relates to a method for anesthetizing an area in a nasal cavity. The method comprises introducing an anesthetic into the nasal cavity using the single-use sprayer described above.

### BRIEF DESCRIPTION OF THE DRAWINGS

FIG. 1 is a schematic of an embodiment of a disposable, single-use sprayer for nasal anesthesia

FIG. 2 a schematic of another embodiment of a disposable, single-use sprayer for nasal anesthesia

FIG. 3 a schematic of yet another embodiment of a disposable, single-use sprayer for nasal anesthesia.

### DETAILED DESCRIPTION

One aspect of the present invention relates to a single-use sprayer for nasal anesthesia. The sprayer includes a single dose of anesthetic and a delivery system adapted to dispense the single dose of anesthetic into a nostril. Comparing to the current multi-dose nasal sprayer, the single-use sprayer prevents patient-to-patient contamination. Moreover, the single-use sprayer is easy to use and allows for self-administration, thus reducing time spent for administering the anesthetic and eliminating time spent cleaning a re-usable device. The sprayer also has a simple, disposable configuration so as to lower the production cost.

The anesthetic can be any topical anesthetic. Examples of topical anesthetic include, but are not limited to, beizocaine, butamben, dibucaine, lidocaine, oxybuprocaine, pramoxine, proparacaine, proxymetacaine, cocaine, and tetracaine (also named amethocaine. In one embodiment, the topical anesthetic is lidocaine, preferably 2% or 4% lidocaine. In another embodiment, the topical anesthetic is a mixture of oxymetazoline and lidocaine, preferably 0.05% oxymnetazoline with 2% or 4% lidocaine. In another embodiment, the topical anesthetic is a mixture of tetracaine aund oxymetazoline, preferably 1% tetracaine in oxymetazoline. In another embodiment, the topical anesthetic is cocaine, preferably 4% cocaine. As used hereinafter, all the percentages refer to weight/volume (w/v) percentages. For example, 2% lidocaine refers to a lidocaine solution containing 20 mg lidocaine per milliliter of the solution.

In certain embodiments, the topical anesthetic is in a pre-mixed liquid form ready to be dispensed into the nasal cavity. The pre-mixed liquid may include the following components: a sterile diluent such as water, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The pH of the pre-mixed liquid can be adjusted with acids or bases, such as hydrochloric acid or sodium hydroxide.

In certain other embodiment, the topical anesthetic is maintained in powder or lyophilized form and is reconstituted before application.

Each nasal sprayer contains a single dosage unit of anesthetic. The dosage unit contains a predetermined quantity of anesthetic calculated to produce the desired anesthetic effect (e.g., numbing the surface of the nasal cavity for a desired period for intended medical procedure) in association with the required pharmaceutical carrier. The specification for the dosage unit forms of the invention are dictated by and directly dependent on the unique characteristics of the active compound and the particular anesthetic effect to be achieved, and the limitations inherent in the art of compounding such an active compound for the treatment of individuals.

The delivery system can be any device suitable for dispensing the single-dose anesthetics into a nasal cavity. In certain embodiments, the delivery system includes a squeeze bottle containing a single dose of anesthetic in solution form. The squeeze bottle is sealed with a tamper proof cap to assure sterility of its content. In other embodiments, the delivery system includes a pressurized container that contains the single-dose anesthetic in a ready to dispense form (e.g., in solution or powder form), and a one-way valve that can be manually open to dispense the anesthetic in a nasal cavity through an applicator. The pressurized container may contain a suitable propellant, e.g., a gas such as carbon dioxide.

In yet other embodiments, the delivery device includes a container that contains the single-dose anesthetic and a manually operable pump for dispensing the anesthetic in a nasal cavity through an applicator.

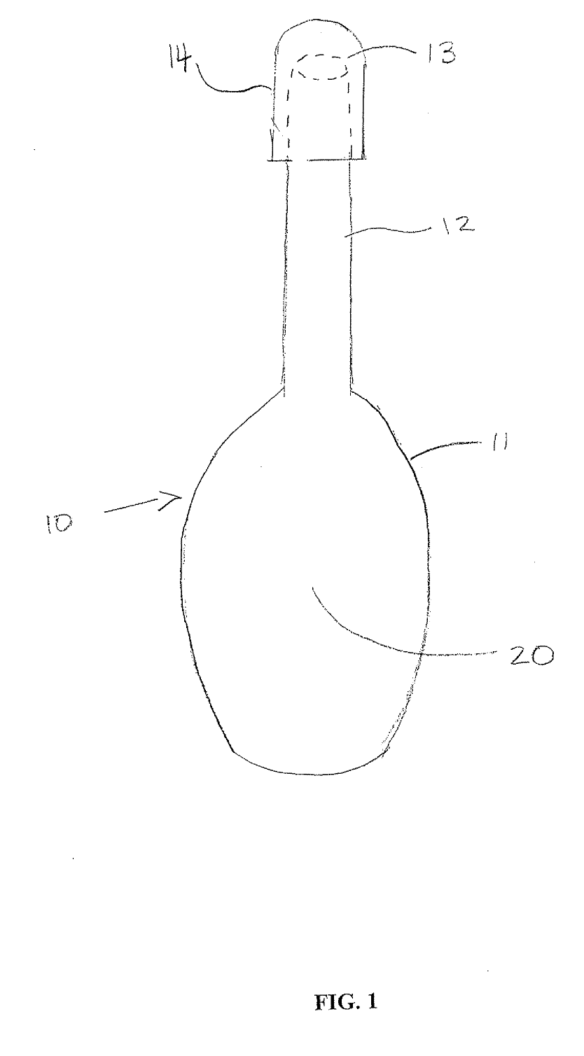

As shown in FIG. 1, an embodiment of the single-use sprayer includes a delivery device 10 having a squeezable container 11 that contains a single dose of anesthetic solution 20. The container 11 has an opening 13 at the tip of a neck 12. The opening 13 is sealed with a tamper proof cap 14. The anesthetic solution 20 is dispensed inside a nasal cavity by simply inserting the neck 12 inside a nostril and squeezing the container 11.

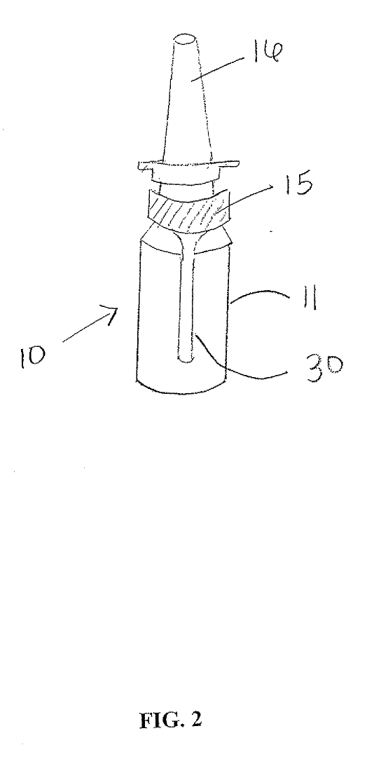

Referring now to FIG. 2, another embodiment of the single-use sprayer includes a delivery device 10 having a container 11 that contains a single dose of anesthetic formulation 30, a dispense control device 15 and an applicator 16. In one embodiment, the container 11 is a pressurized container and the dispense control device is a manually operable one way valve. The anesthetic formulation 30 is released from the pressurized container 11 through the applicator 16 when the valve is in an open position.

In another embodiment, the container 11 is not a pressurized container. The dispense control device 15 is a manually operable pump that pumps the contents of the container into a nostril through the applicator 16.

The applicator 16 can be of any shape and size that are suitable for dispensing the anesthetic into the nasal cavity. In one embodiment, the applicator 16 has a substantially frustoconical profile with curved end edges facilitating insertion into the nostrils.

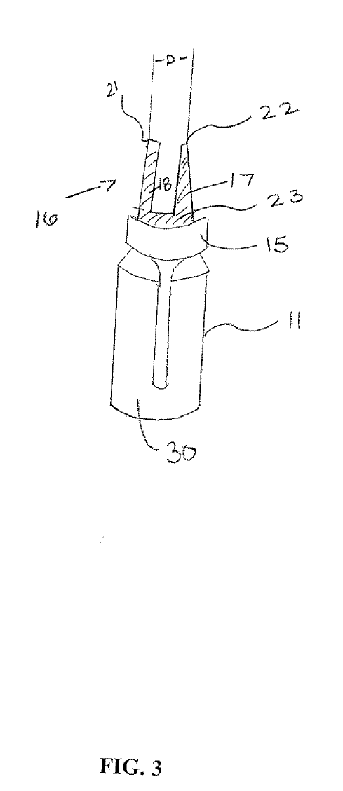

Referring now to FIG. 3, in an embodiment, the applicator 16 comprising two dispensing heads 17 and 18, each of which is provided with at least one expulsion duct. The ends 21 and 22 of the dispensing heads 17 and 18 are separated by a distance (D) that corresponds substantially to the mean spacing between the nostrils. The dispensing heads 17 and 18 join at a base 23 that is coupled to the container 11 through a dispense control device 15. In one embodiment, the container 11 is a pressurized container and the dispense control device 15 a one-way valve. In another embodiment, the container 11 is not pressurized and the dispense control device 15 is a manually operable pump.

Another aspect of the present invention relates to a kit for nasal anesthesia. In one embodiment, the kit includes a sprayer comprising a single dose of anesthetic and a delivery system, and an instruction describing how to use the sprayer. In one embodiment, the single dose of anesthetic is formulated in a solution or suspension form and is ready for dispersion. In another embodiment, the single dose of anesthetic is kept in a lyophilized form and the kit further comprises a reconstitution solution.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

What is claimed is:

1. A single-use sprayer for nasal anesthesia, comprising:
a single dose of anesthetic; and
a delivery system adapted to dispense the single dose of anesthetic into a nostril.

2. The single-use sprayer of claim 1, wherein the anesthetic is a topical anesthetic selected from the group consisting of benzocaine, butamben, dibucaine, lidocaine, oxybuprocaine, pramoxine, proparacaine, proxymetacaine, cocaine, and tetracaine.

3. The single-use sprayer of claim 2, wherein the topical anesthetic comprises lidocaine.

4. The single-use sprayer of claim 2, wherein the topical anesthetic is 2% lidocaine or 4% lidocaine.

5. The single-use sprayer of claim 2, wherein the topical anesthetic comprises a mixture of oxymetazoline and lidocaine.

6. The single-use sprayer of claim 5, wherein the topical anesthetic comprises 0.05% oxymetazoline and 2% lidocaine.

7. The single-use sprayer of claim 5, wherein the topical anesthetic comprises 0.05% oxymetazoline and 4% lidocaine.

8. The single-use sprayer of claim 2, wherein the topical anesthetic comprises a mixture of tetracaine and oxymetazoline.

9. The single-use sprayer of claim 2, wherein the topical anesthetic comprises a mixture of tetracaine and oxymetazoline.

10. The single-use sprayer of claim 9, wherein the topical anesthetic comprises 1% tetracaine in oxymetazoline.

11. The single-use sprayer of claim 2, wherein the topical anesthetic comprises cocaine.

12. The single-use sprayer of claim 11, wherein the topical anesthetic comprises 4% cocaine.

13. The single-use sprayer of claim 1, wherein the delivery system comprises a squeeze bottle containing the single dose of anesthetic in solution form, wherein the squeeze bottle is sealed with a tamper proof cap.

14. The single-use sprayer of claim 1, wherein the delivery system comprises a pressurized container that contains the single-dose anesthetic; a one-way valve; and an applicator, wherein the one-way valve can be manually open to dispense the anesthetic in a nostril through the applicator.

15. The single-use sprayer of claim 14, wherein the applicator has a substantially frustoconical profile with curved end edges to facilitate insertion into the nostril.

16. The single-use sprayer of claim 1, wherein the delivery system comprises a container that contains the single-dose anesthetic; and a manually operable pump for dispensing the anesthetic in a nostril through an applicator.

17. The single-use sprayer of claim 16, wherein the applicator has a substantially frustoconical profile with curved end edges to facilitate insertion into the nostril.

18. A kit for nasal anesthesia, comprising:
at least one single-use sprayer comprising a single dose of anesthetic and a delivery system; and
an instruction describing how to use the sprayer.

19. A method for anesthetizing an area in a nasal cavity, comprising: introducing an anesthetic into the nasal cavity using the single-use sprayer of claim 1.

20. The method of claim 19, wherein the anesthetic is a topical anesthetic selected from the group consisting of benzocaine, butamben, dibucaine, lidocaine, oxybuprocaine, pramoxine, proparacaine, proxymetacaine, cocaine, and tetracaine.
